(19) United States
(12) Patent Application Publication
(10) Pub. No.: US 2023/0364002 A1
(43) Pub. Date: Nov. 16, 2023

(54) CRYSTAL INFUSED FEMININE HYGIENE WASHES

(71) Applicant: Vanessa White, Atlanta, GA (US)

(72) Inventor: Vanessa White, Atlanta, GA (US)

(21) Appl. No.: 17/743,198

(22) Filed: May 12, 2022

(51) Int. Cl. A61K 8/96; A61K 8/04; A61K 8/92; A61Q 19/10

(52) U.S. Cl. CPC A61K 8/965; A61K 8/044; A61K 8/922; A61Q 19/10; A61K 2800/87

(57) ABSTRACT

Systems, apparatuses, and methods of providing feminine hygienic was formulations.

### TECHNICAL FIELD

Embodiments of the invention relate to apparatuses, systems, and methods for feminine hygiene.

### BACKGROUND

The skin of the female genital area contains many nerve cells that are sensitive and may react poorly to abrasive materials and substances. Many different solutions have been proposed and attempted to maintain hydration and protect the skin and underlying nerves from agitation. One of these solutions is a class of products known as intimate washes or feminine washes. These can promote and ensure good feminine genital hygiene and are used to maintain cleanliness of the vulvar-vaginal area.

Feminine washes can detoxify and maintain and promote a natural vaginal flora balance, which helps maintain a state free from irritation, unpleasant odor, and infections. While past feminine washes have included various organic and synthetic substances and additives, none have included crystal infusion. While some designs for crystal infused water bottles for ingestion are known, such bottles may not be appropriately suited for feminine genital application, as they may promote spreading of bacteria to undesirable locations or back into the vessel if used improperly.

Thus, it would be beneficial and needs exist for crystal and other gemstone and rock infused feminine washes in appropriate vessels.

### SUMMARY OF THE INVENTION

This summary is provided to introduce a variety of concepts in a simplified form that is disclosed further in the detailed description of the embodiments. This summary is not intended for determining or limiting the scope of the claimed subject matter.

Crystals, gemstones, and rocks contain various molecular compounds and minerals that can provide health benefits. These compounds and minerals can be extracted, modified, combined, and used for various beneficial effects. Described herein are solutions that include one or more such compounds in the form of feminine washes. These can include other substances and additives in some embodiments.

Systems and apparatuses for vessels including topical feminine washes and associated applicators are also contemplated in various embodiments.

Other objects and advantages of the various embodiments of the present invention will become obvious to the reader and it is intended that these objects and advantages are within the scope of the present invention. To the accomplishment of the above and related objects, this invention may be embodied in the form illustrated in the accompanying drawings, attention being called to the fact, however, that the drawings are illustrative only, and that changes may be made in the specific construction illustrated and described within the scope of this application.

### BRIEF DESCRIPTION OF THE DRAWINGS

A complete understanding of the present embodiments and the advantages and features thereof will be more readily understood by reference to the following detailed description when considered in conjunction with the accompanying drawings wherein:

FIG. 1 illustrates one variation of an apparatus for hygienically dispensing a crystal infused feminine hygiene wash consistent with this disclosure.

### DETAILED DESCRIPTION

The specific details of the single embodiment or variety of embodiments described herein are set forth in this application. Any specific details of the embodiments described herein are used for demonstration purposes only, and no unnecessary limitation(s) or inference(s) are to be understood or imputed therefrom.

Before describing in detail exemplary embodiments, it is noted that the embodiments reside primarily in combinations of components related to devices and systems. Accordingly, the device components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety to the extent allowed by applicable law and regulations. The systems and methods described herein may be embodied in other specific forms without departing from the spirit or essential attributes thereof, and it is therefore desired that the present embodiment be considered in all respects as illustrative and not restrictive. Any headings utilized within the description are for convenience only and have no legal or limiting effect.

Many different embodiments have been disclosed herein, in connection with the above description and the drawings. It will be understood that it would be unduly repetitious and obfuscating to literally describe and illustrate every combination and subcombination of these embodiments. Accordingly, all embodiments can be combined in any way and/or combination, and the present specification, including the drawings, shall be construed to constitute a complete written description of all combinations and subcombinations of the embodiments described herein, and of the manner and process of making and using them, and shall support claims to any such combination or subcombination.

The foregoing is provided for purposes of illustrating, explaining, and describing embodiments of this disclosure. Modifications and adaptations to these embodiments will be apparent to those skilled in the art and may be made without departing from the scope or spirit of this disclosure.

As used herein and in the appended claims, the singular forms “a”, “an”, and “the” include plural referents unless the context clearly dictates otherwise.

Quartz may include silicon dioxide and may be broadly classified as a crystal, gemstone, or rock. Rose quartz exhibits a generally pink or light red hue commonly attributable to the presence of titanium, iron, phosphate, aluminum, or manganese within rose quartz. Rose quartz is a gemstone that carries the energy of love. This gemstone can also bring a person holding, applying, or otherwise using rose quartz feelings of peace, improved compassion and calmness, and self-love. It can also help to dissipate negative emotions.

Rose quartz suspended in solution may improve cleanliness when used as a bodily cleanser as well as provide metaphysical energy to users. Rose quartz may be present in solution in a variety of sizes, arrangements, or amounts such as, but not limited to, gemstones roughly one centimeter in shortest dimension or larger. According to some embodiments, rose quartz may be present in solution in a variety of sizes, arrangements, or amounts such as, but not limited to, gemstones roughly one centimeter in shortest dimension or smaller. According to some embodiments, a crystal infused feminine hygiene wash may include rose quartz suspended in solution such as, but not limited to, purified water.

According to some embodiments, the crystal infused feminine hygiene wash may include at least one of calendula, peppermint oil, lavender oil, sea salt, shea butter, olive oil, chamomile oil, rosemary oil, or aloe vera oil.

According to various embodiments, the crystal infused feminine hygiene wash may be formulated as an infused liquid, gel, lotion, foaming solution, or other form.

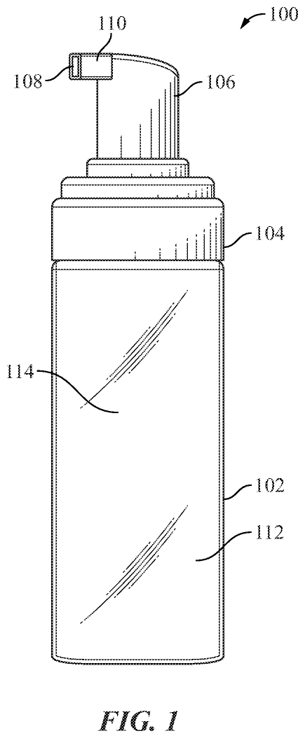

Referring to FIG. 1, a vessel 100 can include an applicator 104 connected to a housing 102 defining a cavity therein to house of the crystal infused feminine hygiene wash 112. The applicator 104 may include a hand or finger pump 106 including an extended neck 110 defining an outlet 108. The pump 106, extended neck 110, and outlet 108 may be constructed and arranged for hygienic dispensing of the crystal infused feminine hygiene wash 112 while minimizing exposure of the outlet 108 to unhygienic user hands, body parts, or environment. The housing 102 and applicator 104 may be connected to one another via a threaded collar or similar arrangement. At least one rose quartz gemstone 114 may be in solution with the of the crystal infused feminine hygiene wash 112. According to some embodiments, the at least one rose quartz gemstone 114 may be roughly one centimeter in shortest dimension or larger or roughly one centimeter in shortest dimension or smaller such that after use or depletion of the crystal infused feminine hygiene wash 112, a user may remove the at least one rose quartz gemstone 114 from the vessel 100. The vessel 100 can house and the pump can dispense an infused liquid, gel, lotion, bubbles, or other form of feminine cleaning product. The vessel 100, including the applicator 104 and the housing 102 may be shaped in various standard, customized, and unique manners, such as normal cylinders, pyramids, spheres, boxes, irregular shapes, or others.

In some embodiments crystals or other rocks, minerals or substances can be housed in specific compartments and/or held in place within the vessel by internal structures coupled with or integral to the vessel such as clamps, housings, bars, or others. In other embodiments, such constituent parts or particles may be free floating within the substance in the vessel. In various embodiments the vessel may be clear or opaque and may allow light in certain wavelengths which can stimulate or promote the desirable properties of the substance. A removable or attached cap or other mechanism can be used to cover the applicator and ensure cleanliness. One or more locking or sealing mechanisms can prevent undesired expression, expulsion, or dispensing of the contained sub stance.

Vessels may be refillable or reusable in some embodiments, while in others they may be disposable. Many materials known and later developed can be used to construct the vessel, such as synthetic compounds like plastic, glass, organic materials such as bamboo, and others. In some embodiments the vessel may be a crystal itself. One or more coupling mechanisms can maintain the connection of the applicator with the vessel, in embodiments where they are not integrated. Various sizes are contemplated, including single use, and much larger.

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the description does not explicitly state, in a particular instance, that such combinations or substitutions are possible. It is explicitly acknowledged that express recitation of every possible combination and substitution is overly burdensome, especially given that the permissibility of each and every such combination and substitution will be readily recognized by those of ordinary skill in the art.

Variation 1 may include a system for feminine genital cleanliness, including a vessel defining a cavity to house a substance; a substance disposed in the vessel; and an applicator coupled to the vessel and operable to dispense the substance in a desired location upon use by a user.

Variation 2 may include a system as in variation 1, wherein the substance includes an infused water.

Variation 3 may include a system as in any of variations 1 through 2, wherein the infused water further includes rose quartz infused water.

Variation 4 may include a system as in any of variations 1 through 3, wherein the substance includes at least powdered rose quartz suspended in a solution.

Variation 5 may include a system as in any of variations 1 through 4, further including at least one rose quartz gemstone disposed within the cavity.

Variation 6 may include a system as in any of variations 1 through 5, wherein the substance includes at least one of calendula, peppermint oil, lavender oil, sea salt, shea butter, olive oil, chamomile oil, rosemary oil, or aloe vera oil.

Variation 7 may include a system as in any of variations 1 through 6, wherein the substance includes at least one of water, rose quartz, calendula, peppermint oil, lavender oil, sea salt, shea butter, olive oil, chamomile oil, rosemary oil, and aloe vera oil.

Variation 8 may include system for feminine genital cleanliness, including a housing defining a cavity; an applicator including a pump, an extended neck defining an outlet, and a collar constructed and arranged to mechanically connect to the housing; and a crystal infused feminine hygiene wash disposed within the cavity and in fluid communication with the cavity, the pump, the extended neck, and the outlet.

Variation 9 may include a system as in variation 8, wherein the crystal infused feminine hygiene wash includes at least powdered rose quartz suspended in a solution.

Variation 10 may include a system as in any of variations 8 through 9, wherein the crystal infused feminine hygiene wash includes rose-quartz infused water.

Variation 11 may include a system as in any of variations 8 through 10, further including at least one rose quartz gemstone disposed within the cavity.

Variation 12 may include a system as in any of variations 8 through 11, wherein the crystal infused feminine hygiene wash includes rose-quartz infused water.

Variation 13 may include a system as in any of variations 8 through 12, wherein the crystal infused feminine hygiene wash includes at least one of calendula, peppermint oil, lavender oil, sea salt, shea butter, olive oil, chamomile oil, rosemary oil, or aloe vera oil.

Variation 14 may include a system as in any of variations 8 through 13, wherein the crystal infused feminine hygiene wash includes at least one of water, rose quartz, calendula, peppermint oil, lavender oil, sea salt, shea butter, olive oil, chamomile oil, rosemary oil, and aloe vera oil.

Variation 15 may include a system for feminine genital cleanliness, including a housing defining a cavity; an applicator including a pump, an extended neck defining an outlet, and a collar constructed and arranged to mechanically connect to the housing, wherein the cavity, the pump, the extended neck, and outlet are in fluid communication with one another; at least one rose quartz gemstone disposed within the cavity; and a crystal infused feminine hygiene wash including rose quartz in solution disposed within the cavity and in fluid communication with the cavity, the pump, the extended neck, and the outlet.

Variation 16 may include a system as in variation 15, wherein the crystal infused feminine hygiene wash includes at least one of calendula, peppermint oil, lavender oil, sea salt, shea butter, olive oil, chamomile oil, rosemary oil, or aloe vera oil.

Variation 17 may include a system as in any of variations 15 through 16, wherein the housing is a generally cylindrical bottle.

Variation 18 may include a system as in any of variations 15 through 17, wherein the pump includes a foaming pump.

Variation 19 may include a system as in any of variations 15 through 17, wherein the applicator is constructed and arranged for hygienic dispensing of the crystal infused feminine hygiene wash while minimizing exposure of the outlet to unhygienic user hands, body parts, or environment.

Variation 20 may include a system as in any of variations 15 through 19, further including a removable cap constructed and arranged to attach to and cover at least a portion of the applicator.

In many instances entities are described herein as being coupled to other entities. It should be understood that the terms “coupled” and “connected” (or any of their forms) are used interchangeably herein and, in both cases, are generic to the direct coupling of two entities (without any non-negligible (e.g., parasitic) intervening entities) and the indirect coupling of two entities (with one or more non-negligible intervening entities). Where entities are shown as being directly coupled together or described as coupled together without description of any intervening entity, it should be understood that those entities can be indirectly coupled together as well unless the context clearly dictates otherwise.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the inventive scope of the claims by features, functions, steps, or elements that are not within that scope.

An equivalent substitution of two or more elements can be made for any one of the elements in the claims below or that a single element can be substituted for two or more elements in a claim. Although elements can be described above as acting in certain combinations and even initially claimed as such, it is to be expressly understood that one or more elements from a claimed combination can in some cases be excised from the combination and that the claimed combination can be directed to a subcombination or variation of a subcombination.

It will be appreciated by persons skilled in the art that the present embodiment is not limited to what has been particularly shown and described herein. A variety of modifications and variations are possible in light of the above teachings without departing from the following claims.

What is claimed is:

1. A system for feminine genital cleanliness, comprising:
a vessel defining a cavity to house a substance;
a substance disposed in the vessel; and
an applicator coupled to the vessel and operable to dispense the substance upon use by a user.

2. The system of claim 1, wherein the substance comprises an infused water.

3. The system of claim 2, wherein the infused water further comprises rose quartz infused water.

4. The system of claim 1, wherein the substance comprises at least powdered rose quartz suspended in a solution.

5. The system of claim 1, further comprising at least one rose quartz gemstone disposed within the cavity.

6. The system of claim 1, wherein the substance comprises at least one of calendula, peppermint oil, lavender oil, sea salt, shea butter, olive oil, chamomile oil, rosemary oil, or aloe vera oil.

7. The system of claim 1, wherein the substance consists essentially of at least one of water, rose quartz, calendula, peppermint oil, lavender oil, sea salt, shea butter, olive oil, chamomile oil, rosemary oil, and aloe vera oil.

8. A system for feminine genital cleanliness, consisting essentially of:
a housing defining a cavity;
an applicator comprising a pump, an extended neck defining an outlet, and a collar constructed and arranged to mechanically connect to the housing; and
a crystal infused feminine hygiene wash disposed within the cavity and in fluid communication with the cavity, the pump, the extended neck, and the outlet.

9. A system for feminine genital cleanliness as in claim 8, wherein the crystal infused feminine hygiene wash comprises at least powdered rose quartz suspended in a solution.

10. A system for feminine genital cleanliness as in claim 8, wherein the crystal infused feminine hygiene wash comprises rose-quartz infused water.

11. A system for feminine genital cleanliness as in claim 8, further comprising at least one rose quartz gemstone disposed within the cavity.

12. A system for feminine genital cleanliness as in claim 8, wherein the crystal infused feminine hygiene wash consists essentially of rose-quartz infused water.

13. A system for feminine genital cleanliness as in claim 8, wherein the crystal infused feminine hygiene wash comprises at least one of calendula, peppermint oil, lavender oil, sea salt, shea butter, olive oil, chamomile oil, rosemary oil, or aloe vera oil.

14. A system for feminine genital cleanliness as in claim 8, wherein the crystal infused feminine hygiene wash consists essentially of at least one of water, rose quartz, calendula, peppermint oil, lavender oil, sea salt, shea butter, olive oil, chamomile oil, rosemary oil, and aloe vera oil.

15. A system for feminine genital cleanliness, consisting essentially of:
a housing defining a cavity;
an applicator comprising a pump, an extended neck defining an outlet, and a collar constructed and arranged to mechanically connect to the housing, wherein the cavity, the pump, the extended neck, and outlet are in fluid communication with one another;
at least one rose quartz gemstone disposed within the cavity; and
a crystal infused feminine hygiene wash comprising rose quartz in solution disposed within the cavity and in fluid communication with the cavity, the pump, the extended neck, and the outlet.

16. A system for feminine genital cleanliness as in claim 15, wherein the crystal infused feminine hygiene wash comprises at least one of calendula, peppermint oil, lavender oil, sea salt, shea butter, olive oil, chamomile oil, rosemary oil, or aloe vera oil.

17. A system for feminine genital cleanliness as in claim 15, wherein the housing is a generally cylindrical bottle.

18. A system for feminine genital cleanliness as in claim 15, wherein the pump comprises a foaming pump.

19. A system for feminine genital cleanliness as in claim 15, wherein the applicator is constructed and arranged for hygienic dispensing of the crystal infused feminine hygiene wash while minimizing exposure of the outlet to unhygienic user hands, body parts, or environment.

20. A system for feminine genital cleanliness as in claim 15, further comprising a removable cap constructed and arranged to attach to and cover at least a portion of the applicator.
